(19) United States
(12) Patent Application Publication
(10) Pub. No.: US 2017/0307596 A1
(43) Pub. Date: Oct. 26, 2017

(54) Methods for Conducting Stimulus-Response Studies with Induced Pluripotent Stem Cells Derived from Perinatal Cells or Tissues

(71) Applicant: Coyne IP Holdings, LLC, Smyrna, GA (US)

(72) Inventors: Shawn T. Coyne, Smyrna, GA (US); Kevin P. Coyne, Atlanta, GA (US)

(21) Appl. No.: 15/518,316

(22) PCT Filed: Oct. 15, 2015
(86) PCT No.: PCT/US15/55637

§ 371 Date: Apr. 11, 2017

Related U.S. Application Data
(60) Provisional application No. 62/064,067, filed on Oct. 15, 2014.

(51) Int. Cl. G01N 33/50; C12N 5/074; C12N 5/0735; A61K 35/12; C12Q 1/02; C12N 5/00

(52) U.S. Cl. CPC G01N 33/5073; C12N 5/0606; A61K 35/12; C12N 5/0696; C12N 5/00; C12Q 1/02

(57) ABSTRACT

Methods are provided herein for conducting stimulus-response studies on iPSCs, or cells derived from iPSCs, that have been derived from perinatal cells collected from donors under null-exposome conditions. In some embodiments, multiple donors are involved. In other embodiments, the iPSCs are differentiated. The use of induced pluripotent stem cells (iPSCs) derived from cells that originate from neonates enable a scientist to largely remove the influences of age and environmental exposures, allowing a more targeted analysis of the direct interaction between a stimulus and a test subject. Furthermore, use of iPSCs derived from cells originating from multiple donors enable the scientist to obtain more precise measurements of the role of genetic differences in determining responses to a given stimulus than the use of other test materials, by eliminating the vast majority of the differential influences of age and environment among test subjects.

### FIELD OF THE INVENTION

The present application is directed to methods for conducting stimulus-response studies using induced pluripotent stem cells, wherein the influence of environmental factors on the behavior of those cells has been minimized, or using cells differentiated therefrom.

### BACKGROUND

The scientific community has long been interested in the underlying causes of biological responses of cells and tissues to various stimuli, such as the administration of pharmaceuticals, and exposure to chemicals, bacteria and viruses. Scientists have recognized that the following factors play key roles in the response: the nature of the stimulus; the genetic profile of the test subject; the age of the test subject; and the unique history of physical exposures, such as diet, disease, chemicals, and environmental insults, that the subject has endured throughout its lifetime. However, scientists have had difficulty sorting out the contributions of the various factors because any available cells or tissues from a test subject, and the associated testing methodologies, inevitably exhibited an idiosyncratic combination of all four factors.

Scientists in certain fields such as pharmaceuticals and environmental toxicology have been particularly interested in determining the underlying causes of differences in the biological responses to a given stimulus among multiple test subjects. Scientists have taken particular interest in discovering the role that genetic diversity plays in differences in biological responses. However, discerning that role is difficult. To succeed, scientists must overcome four problems simultaneously.

Problem 1: Scientists need a large sample base, as the diversity of genetic profiles is quite extensive, and there is usually no pre-existing guidance as to which precise subset of genes is involved in the response to a particular stimulus.

Problem 2: Scientists need a test platform (in the form of cells, tissues, organs, or whole animals or human beings) whose response, when challenged in a stimulus-response experiment (either in vivo or in vitro), is predictive of the response of the test subject as a whole organism in at least one relevant way.

Problem 3: Scientists need to conduct the stimulus-response experiment with adequate controls, such that variation is due to genetic differences, rather than variations in the experimental procedure.

Problem 4: Scientists must effectively overcome the “noise” of extraneous factors. That is, they must deal with the fact that the biological reactions of the cells or tissues from any one subject depend not only on the specific genetic profile of that subject, but also on the age of that subject, and that subject's unique history of exposures to various external forces (such as diet, disease, chemicals, and environmental pollution).

The nature of the test platform (i.e., cells, tissues, organs, or whole animals or human beings) and the test subjects themselves plays a profound role in the degree to which the scientist can overcome each of these problems.

To date, the scientific community's primary tool for studying the underlying causes of biological reactions has been large scale gene association studies based on epidemiological data. This approach can adequately address Problems 1 and 2 (large sample base and test platform having a response predictive of the response of the test subject), but falls short on Problems 3 and 4 (minimal variations in experimental procedure and elimination of “noise” of extraneous factors). Idiosyncratic behaviors of test subjects, such as failing to take the candidate drug or placebo as directed, or failing to accurately report their biological responses to the drug, are well documented as being widespread, and as interfering with the experimental integrity. Further, every test subject has a specific age, almost always different than that of other subjects, and a unique history of exposure to external forces. Consequently, statistical finding about the roles played by genetics are often faint, showing only small impacts.

In response, some scientists have chosen to base stimulus-response experiments on cellular- or tissue-based models, using primary cells or tissues taken from living humans or cadavers. These samples are difficult and expensive to obtain, thus falling victim to Problem 1—the inability to gather samples from a large enough pool of donors to satisfy the statistical requirements of genetic studies. In addition, these models fail to overcome Problem 4 (i.e., the interference of age and exposure history).

In an alternative attempt to overcome Problem 1, some scientists have tried using tumoric or engineered cells. These samples run the risk of falling victim to Problem 2 (i.e., the response of the cells used in the test being unrepresentative of the response of the organism they are supposed to represent), as well as Problem 4 (i.e., the interference of age and exposure history).

Recently, scientists have begun using induced pluripotent stem cells (iPSCs), or functional cells derived from iPSCs, in stimulus-response studies. Such cells can overcome Problems 1 (need for large sample base), 2 (need for representative test platform) and 3 (need for experimental procedure controls). First, they can be obtained from large numbers of subjects. Second, as long as the cells have been completely reprogrammed to a pluripotent state via a non-integrating method, the response can be correlated with clinical results with the genetic profile intact. And third, the stimulus-response experiment can be controlled.

However, Problem 4 has yet to be overcome. The stimulus-response experiments conducted to date, using iPSCs reprogrammed from adult somatic cells, embed the age of the donor of the somatic cell that has been reprogrammed, as well as the unique exposure history of that donor. Because all of the donors in such experiments to date have been adults, the variability in age and exposure history is extensive.

It is well established that a test subject's age affects the biological processes of the body (Finch 1990, Vijg 2000, Ono et al 2002, Trifunovia and Larssen 2008). This is problematic because the scientific community knows neither (1) all the effects that age has at the cellular or tissue level, nor (2) how to artificially reverse or eliminate the effects it does know about. Therefore, attempts to minimize the impact of age cannot be accomplished through efforts to remove the effects of age already present in a cell.

Similarly, it is well established in the scientific literature that a wide variety of environmental exposures such as disease, pollutants, pharmaceutical or recreational drugs, and even diet can lead to permanent changes in reactions to subsequent stimuli. But, as with age, scientists know neither the full extent and nature of these effects, nor how to remove these effects from cells or tissues.

Therefore, what is needed is a method for obtaining and utilizing iPSCs from samples of donors who do not embed the effects of aging or different environmental exposures, thus eliminating the potential for differences in these factors to differentially affect the stimulus-response behavior.

### BRIEF SUMMARY OF THE INVENTION

Methods are provided herein for conducting stimulus-response studies on iPSCs, or cells derived from iPSCs, that have been derived from perinatal cells collected from donors under null-exposome conditions. In some embodiments, multiple donors are involved. Further, in some embodiments, the iPSCs are differentiated into functional cell types, such as cardiomyocytes or neurons..

In accordance with the method, null-exposome conditions appropriate to the study are identified, the type of perinatal cell to be collected is specified, optimal donor selection conditions are determined, cell collection procedures are established and potential donors who can meet the null-exposome conditions required by the study are identified.

Scientifically useful measurements result when the identified cells are actually collected from the identified donors in the prescribed way, converted to iPSCs, optionally differentiated into any of a variety of functional cells types, optionally formed into tissues, the stimulus applied, and the resulting biological response measured and analyzed. Optionally, practitioners may cryopreserve and later thaw the cells (or their progeny or any derivative cells) at any appropriate point in the process.

Four factors interact to determine the biological response of individual cells or tissues from a particular test subject, or sample, to a particular stimulus: the nature of the stimulus; the genetic profile of the test subject; the age of the test subject; and the unique history of physical exposures, such as diet, disease, chemicals, and environmental insults, that the subject has endured throughout its lifetime. The use of induced pluripotent stem cells (iPSCs) derived from perinatal cells collected from a donor under null-exposome conditions enable the practitioner to largely remove the influences of age and environmental exposures, allowing a more targeted analysis of the direct interaction between a stimulus and a test sample. Further, use of such iPSCs derived from cells originating from perinatal cells from multiple donors can enable the practitioner to obtain more precise measurements of the role of genetic differences in determining differential responses to a given stimulus than the use of other test materials, by eliminating the vast majority of the differential influences of age and environment among test subjects.

### DETAILED DESCRIPTION OF THE INVENTION

Methods for conducting stimulus-response studies are provided herein in which induced pluripotent cells (iPSCs), or cells derived therefore, are employed. The iPSCs used in the method have been produced from perinatal cells, obtained from pre-selected donors under null-exposome conditions. In some embodiments, multiple donors are involved. Further, in some embodiments, the iPSCs are differentiated into functional cell types.

Prior to conducting the study, the following three independent actions are determined: (a) null-exposome conditions appropriate to the study must be specified; (b) the type of perinatal cell to be obtained must be determined as well as the donor selection conditions and cell collection procedures; and (c) potential donor, or donors, who meet the null-exposome conditions demanded by the study are identified.

Scientifically useful information results from use of the method provided herein when the particular type of perinatal cells are collected from the identified donors under null-exposome condition, converted to iPSCs, differentiated (if appropriate to the study at hand) into one or more of a variety of functional cells types, combined into tissues (again, if appropriate), the stimulus applied, and the resulting biological response measured and analyzed. Optionally, the cells, or their progeny or any derivative cells, are cryopreserved and subsequently thawed at any appropriate point in the process.

Definitions

The term “perinatal period”, as used herein, means the life phase surrounding the time of birth, specifically, the time immediately before and after birth, such as from the beginning of the second trimester of gestation to six months post-birth or less.

The term “perinatal cell”, as used herein, means any cell that can be genetically identified as having originated from a donor (human or other mammal) during the perinatal period. Perinatal cells include, but are not limited to: umbilical cord blood cells or umbilical cord tissue of any kind, including any cells derived therefrom; peripheral blood cells; amniotic fluid stem cells (whether found directly in amniotic fluid or taken from the placenta or elsewhere); cells found in neonatal urine; cells obtained from skin (including dermal fibroblasts) or hair; and cells obtained through a swab of the cheek or any alimentary passage of the donor.

The term “exposome”, as used herein, means the cumulative measure of environmental influences of biological responses throughout the lifespan of a human or other mammal, including but not limited to exposures from behavior, diet, disease, bacteria, viruses, pharmaceuticals, chemicals, and the environment, as well as any previous endogenous responses to these external stimuli which permanently affect subsequent biological responses. Thus, this definition includes not only the direct impact of external challenges, but also the indirect effects of such challenges, such as epigenetic changes, scarring, DNA mutation or larger chromosomal damage.

The term “null-exposome conditions”, as used herein, means conditions wherein a cell, set of cells, or tissues constructed entirely or in part from those cells, in which the researcher has taken measures of any kind to restrict, reduce or eliminate impacts on the experimental response of those cells (within the acceptable tolerances prescribed for that experiment) due to either age-related effects of the cells themselves, or exposure of the cells to the exposome, wherein such actions may include, but are not limited to, the utilization of selection criteria, inducements to the mother to restrict behavior during pregnancy or birth, and actions that shield the source cells from exposures during collection and processing. In situations where multiple test subjects are involved, null-exposome conditions include cells that have had some exposure to the exposome, provided that: (1) the exposure is judged be likely to have minimal impact on responses in the experiment or study under investigation; (2) steps have been taken to minimize any differences in exposures among test subjects; and (3) any remaining differences in exposures among the donors are explicitly judged to fall within acceptable tolerances prescribed for that experiment or study.

The terms “stimulus or stimuli”, as used herein, means any external physical material or force that is applied alone, in plurality, or in any combination, and that may cause a reaction in the behavior or structure of the cells or tissues under investigation. Examples of stimuli include, but are not limited to, chemicals, including pharmaceutical compounds and industrial chemicals; biological agents such as bacteria, viruses, molds, mycoplasma, allergens or other pathogens; light; heat; sound; atmospheric pressure; electrical impulses; physical trauma; radiation; and any form of physical stress, including that resulting from externally induced physical activity of the cells themselves.

Method Development

Applicants deduced that, in the task of dissecting the causes of biological responses to stimuli among one or more test subjects: (1) the only method to disaggregate genetic effects from age and exposure effects would be to conduct the stimulus-response study on a platform built from test subjects who are of identical ages, and who are definitively known to have been only minimally exposed to only benign environments that are highly similar among all donors; and (2) the only potential test subjects who meet these conditions from a practical standpoint are cells of donors that are fetuses or neonates.

Applicants further deduced that: (i) most cell types of fetuses or neonates (e.g., embryonic stem cells, amniotic fluid cells, primary cells) contain limitations on their suitability for stimulus-response studies; (ii) iPSCs derived from perinatal cells are potentially highly suitable for stimulus-response studies; (iii) however, a practitioner's decision to use a perinatal-cell-derived iPSC will not, in and of itself, result in the null-exposome conditions required to eliminate the impacts of age and environmental exposure from the cells, and hence from the stimulus-response study—additional decisions and controls are required; and (iv) three sets of interdependent actions are required to achieve the null-exposome-conditions in stimulus-response studies.

Each of these deductions (i) through (iv) is discussed in detail below.

(i) Most cell types of fetuses or neonates (e.g., embryonic stem cells, first trimester amniotic fluid cells, primary cells) contain limitations on their suitability for stimulus-response studies. Applicants reviewed the suitability of several cell types: embryonic stem cells; direct use of primary cells from the period prior to the perinatal period; direct use of cells derived from amniotic fluid prior to the perinatal period; and the direct use of primary perinatal cells (in addition to the use of iPSCs derived from perinatal cells, as discussed below under item (ii)).

Embryonic stem cells (ESCs) cultivated in vitro, and cells derived therefrom, can be of limited usefulness as the basis for stimulus-response studies. They benefit from being the only cells that are of “theoretical-zero age”. However, while ESCs could be useful for single-donor experiments, they are not suitable for multi-donor experiments because, given the ethical issues involved, it is highly unlikely that a practitioner could develop a cohort of ESCs from a sufficiently large number of donor embryos to enable any statistically-meaningful analysis of the impact of genetics on differential responses.

Direct use of primary cells from the period prior to the perinatal period could not be obtained without a degree of invasiveness to the fetus wherein the ethical, safety and practical issues would be prohibitive.

Direct use of cells from amniotic fluid collected prior to the perinatal period (i.e., use of the cells themselves, or use of cells differentiated directly from such cells, rather than via transfection into iPSCs followed by differentiation into functional cell types as described in this invention) suffers from multiple issues. First, while such cells might hold some advantage over perinatal cells by virtue of being younger, practitioners find that such cells have been shown to be unable to differentiate into a number of key functional cell types, such as mature cardiomyocytes. Further, comparability among test subjects is an issue, for at least three reasons: (1) obtaining cells of the precisely same age would be extremely difficult (note that any deviation in age—for example, five days—represents a significant variation when dealing with extremely young cells, such as first trimester cells that are on average 45 days old); even if one could coordinate a large number of mothers to agree to have the amniocentesis procedure performed at the precisely “same” time (relative to conception), the practitioner would be unable to specify that time for each mother, because the exact date of conception is rarely known, so age-conformity would be approximate at best. (2) No two fetuses develop at precisely the same rate, particularly during the first trimester; therefore, at any specified date after conception, different fetuses are at different points in their development—creating an obvious (but unanalyzable) differential in the likely impact of exposures, since the same exposure has been shown to have different impacts at different points in the development of a fetus. (3) There are known to be many types of “stem cells” in amniotic fluid, and there has been little study of the diversity among fetuses in the character of any one particular amniotic fluid cell type at a single point in time after conception. Thus, a quest to use the youngest possible cell comes at the expense of the introduction of significant “noise” in achieving equivalent null-exposome conditions across test subjects.

Primary perinatal cells, as well as other prenatal or near-natal cells, are generally poor candidates for age-and-exposure-neutral stimulus-response studies. Primary tissues of healthy babies are of limited use, because of the ethical issues involved, as well as the fact that the supply of tissues from a single subject is limited in both supply and time, thus making cross-experimental comparisons difficult. Tissues from stillborn babies or aborted fetuses are subject to these same limitations, and have, by definition, either developed abnormally in some way, or been subjected to undesirable or lethal environmental exposures.

(ii) In contrast to all of the above cell types and sources, iPSCs developed from perinatal cells are highly suitable for stimulus-response studies.

Perinatal cells taken immediately at the time of birth offer the best practical choice for achieving a zero-age condition that is functionally equivalent across test subjects. Science has long accepted the premise that neonates resulting from full-term (but not post-mature term) births are essentially age equivalent and developmentally equivalent. The intellectual basis for this premise is that mother's body and the fetus typically coordinate to initiate the birth process at the point when that particular fetus is developmentally ready for birth. While that time might come earlier or later (relative to the time of conception) depending on the fetus, the developmental age of each neonate is considered equivalent at birth—that is why the entire fields of childhood development and medicine rely on chronological age (i.e., time since birth) as the central age parameter to be referenced. Therefore, in order to minimize the impact of age, the practitioner may obtain perinatal cells as close to the moment of birth as is practical for all test subjects in the cohort.

The use of iPSCs created from materials that can be collected non-invasively from neonates (i.e., perinatal cells) for use in stimulus-response studies could overcome all four problems described above. First, the required source material can be obtained and utilized from large numbers of donors cost effectively, since the collection can be non-invasive, and the resulting iPSCs can replicate indefinitely (hence being available for use in multiple experiments). Second, responses of iPSCs (and their derivative cells) have been shown to be relevantly predictive of clinical results. Third, tight experimental controls can be enforced, as the experiments are conducted in vitro rather than in vivo. Fourth, by obtaining donor cells at birth only from neonates whose mothers' environmental exposures and personal consumption behaviors have been similar during gestation, the practitioner can both eliminate age differences among the test subjects and reduce exposure differences to a small fraction of those experienced among adult donors.

The potential for use of iPSCs derived from perinatal cells for age-and-exposure-neutral stimulus-response studies has not been previously considered. A number of research groups have demonstrated that iPSCs can be derived successfully from a variety of perinatal tissues and cell types (for example, see Cai et al. 2010, Connell et al. 2013, Haase et al. 2009, Jiang et al. 2014). However, none of these practitioners have described the use of perinatal cell-derived iPSCs (or downstream cells derived therefrom) for this purpose. In fact, the literature fails to even show recognition of the unique characteristic of these cells (i.e., the lack of age and the potential similarity of minimal environmental exposures in the womb) that is necessary to realize the potential use of these cells in this context.

(iii) However, a practitioner's decision to use a perinatal-cell-derived iPSC will not, in and of itself, result in the null-exposome conditions required to eliminate the impacts of age and environmental exposure from the cells, and hence from the stimulus-response study—additional decisions and controls are required. Therefore, the present method extends beyond the mere recognition of the appropriate characteristics of perinatal cell-derived iPSCs for the purpose of conducting stimulus-response studies. Rather, the scientist must explicitly select and enforce a number of additional choices and conditions. Specifically, achieving null-exposome conditions requires the scientist to exercise control over both (1) the choice of donors, based on narrowing the range of potential exposures of both mother and fetus during gestation; and (2) the precise timing (relative to the moment of birth) of the cell collection process.

Regarding narrowing the range of potential exposures of both mother and fetus during gestation, it is important to note that the use of any perinatal cells introduces the potential for impact from the exposome on the mothers, and hence on the mothers' wombs, and thus introduces the potential for donor-to-donor differences. Fortunately, basic female physiology provides significant protection from such differences, in that reproductive biology favors the health of the fetus over that of the mother such that the mother typically absorbs variations in her environment, passing on to the fetus the required nutrients and environment (to the degree that her body is able). Nevertheless, a practitioner's choices can further reduce any differentials in the womb environment, by employing strict inclusion/exclusion criteria based on limiting the variation in the mother's ‘own environment, and on the environment her own behavioral choices impose on the fetus. Such criteria include, but are not limited to, excluding test subjects (1) if the mother has engaged in activities known to produce harmful effects on the fetus, such as excessive smoking, drinking or the use of recreational drugs; (2) if the mother has taken certain prescription drugs during pregnancy; (3) if the mother has experienced any trauma that might impact the womb; (4) if the neonate was born either prematurely or post-mature; (5) if the mother was exposed to radiation, or high levels of pollution (airborne, ground-water, or background based); or (6) if the mother was exposed to toxins. In addition, the practitioner can place further restrictions to ensure greater consistency, such as restricting the pool of donor neonates to those born in a particular locality and within a short time period, which would serve to homogenize the external pollution environment experienced by their mothers during pregnancy. The practitioner can even exercise some control of maternal diet during gestation, through exclusion criteria or behavioral agreements with the mothers.

Regarding the precise timing (relative to the moment of birth) of the cell collection process, it is well documented that neonates can be permanently affected by certain exposures to chemicals and diseases. Modern birth practices often introduce such exposures within minutes of birth—for example, neonates born in many hospitals in the United States are injected with a vaccine for Hepatitis B in the delivery room itself. Further immunizations occur soon thereafter. Therefore, to achieve null-exposome conditions, the practitioner must either obtain the specified perinatal cells from all donors prior to when that particular perinatal cell would be affected by such exposures, or explicitly conclude that (for purposes of the stimulus-response experiment in question) the particular exposures that occur prior to collection will not affect the biological responses to the stimulus. And, in either case, the practitioner must control circumstances to ensure that all donors' exposure experiences lie within the tolerance specified for the study.

(iv) Three sets of interdependent actions are required to achieve the null-exposome-conditions in stimulus-response studies. They are: (a) Specify the null-exposome conditions appropriate to the study; (b) Specify the type of perinatal cell to be obtained, the donor selection conditions, and cell collection procedures accordingly; and (c) Identify potential donor(s) who can meet the null-exposome conditions demanded by the study.

As noted earlier, scientifically useful measurements result when pre-identified cells are actually collected from the predetermined donors in the prescribed way, converted to iPSCs, differentiated (if appropriate to the experiment at hand) into any of a variety of functional cells types, combined into tissues (again, if appropriate), the stimulus applied, and the resulting biological response measured and analyzed. Optionally, practitioners may cryopreserve and later thaw the cells, or their progeny or any derivative cells, at any appropriate point in the process, using methods known to those skilled in the art.

Stimulus-Response Method

Accordingly, methods are provided for determining a response of a test sample to a stimulus by applying the stimulus to one or more test samples derived from one or more perinatal cells isolated from one or more donors under predefined null-exposome conditions and detecting the response by the test sample to the stimulus. Any differential response among the donors is attributed to the differences in the genetics of the donors rather than the age or environmental condition to which the donors were subjected prior to sample collection.

In accordance with the method, the test sample is one or more induced pluripotent stem cells or a cell or cells differentiated therefrom, or a tissue formed from the differentiated stem cells. Methods for producing induced pluripotent stem cells, differentiating stem cells and forming tissues from differentiated stem cells are known to those skilled in the art. Such known methods are used herein. Also in accordance with the method, a detectable response, either positive or negative when compared to a control, indicates that the stimulus has an effect on the test sample. A lack of detectable response when compared to a control indicates that the stimulus has no effect or an inconsequential effect on the test sample. Methods for detecting the response of a test sample to a stimulus are known to those skilled in the art. Such known methods are used herein.

Differences in the effect of the stimulus among the donors due to genetic differences may be determined by collecting test samples from two or more donors and preforming the method as described herein.

Identification of Null-Exposome Conditions Appropriate to the Study

The practitioner begins with a prescriptive description of the types of cells and conditions that are appropriate to the experiment. This description is then translated into requirements for the gestational and birthing environment, as detailed below.

The description consists of: (a) requirements regarding the genetic profile and health at birth of the donor neonate; (b) requirements regarding the mother's health history, particularly during pregnancy; (c) requirements regarding the mother's exposure to environmental factors (before and/or during pregnancy) that could affect the long term health of the donor neonate, and consequently the epigenetic profile of the donor neonate's cells; (d) requirements regarding exposure of the donor baby (in utero) to adverse substances or conditions; and (e) requirements regarding exposures of the donor neonate post-birth (if the experiment-appropriate cells will be collected post-birth).

No cell, not even a perinatal cell, is perfectly free of environmental exposures. Fortunately, however, if the exposures experienced by a particular subset of perinatal cells from a particular donor are known from prior research (or, less ideally, are believed) to not significantly affect the results of a particular design of stimulus-response experiment, then that subset of cells can, for practical purposes, be considered to be null-exposome condition cells. Similarly, if a particular subset of perinatal cells taken from multiple donors have experienced practically identical exposures of the type that might affect results, then the cells can be considered null-exposome condition cells within the confines of that particular experiment (although comparative results across donors should be reported as pertaining only to the specific set of environmental conditions common to all the donors).

This construct provides guidance to the scientist when developing the specifications for the source cell. Based on the specific nature of the stimulus to be applied and the response to be measured, the scientist must determine the comprehensive list of types (and associated extents) of exposures that are known (or believed) to affect experimental results, and design the specifications for the source cell accordingly. For example, if the stimulus is to be a pharmaceutical compound and the response to be measured is the change in production by the iP SC (or its derivative cells) of a particular protein, then the specifications will likely include a prohibition of any exposures that directly increase or decrease production of that protein by the source cells in utero, such as certain pharmaceuticals, recreational drugs, etc.

Specification of Perinatal Cell Type, Donor Selection Conditions and Cell Collection Procedures

The specification of the required null-exposome conditions guides the selection of perinatal cell type to be obtained, the identification of eligible donors and the cell collection procedures.

The perinatal cell type to serve as the source of the iPSCs is predetermined because, even within the context of a single donor, the various types of perinatal cells may experience different exposures. For example, cells from amniotic fluid taken at birth (which are usually obtained during pre-planned Caesarian sections) have usually been exposed to the mother's blood as a result of the bleeding that takes place from the Caesarian incision, whereas cells derived from amniotic fluid obtained through third trimester amniocentesis have not been so exposed. As another example, cells taken from skin fibroblasts post-birth have potentially been exposed during the birthing process to any vaginal infections of the mother (such as sexually transmitted diseases, or Group B strep infection), whereas cells from cord blood are less likely to have been so exposed.

The specification of eligible donors serves to eliminate two sets inappropriate donors: those whose underlying health condition would render any iPSCs derived from its cells as an inaccurate model, and those who have experienced a proscribed exposure. Examples of the first category might include, but are not limited to: donors with known genetic defects or damage; donors whose families have a history of genetically based diseases that bear on the experiment in question; or donors who were born significantly prematurely. Examples of the second category might include, but are not limited to: donors whose mothers ingested proscribed substances during pregnancy, such as recreational drugs, certain prescription drugs, or excessive alcohol; donors whose mothers engaged in proscribed activities during pregnancy, such as excessive smoking; or donors whose mothers were exposed to radiation or high levels of toxic pollution.

The conditions and procedures of cell collection can also have an effect on the ability to meet the designated null-exposome conditions, and therefore must be specified. Obvious examples include ensuring the sterility of collections, transportation, and processing procedures, but there are less obvious considerations as well. For example, the precise timing of the tissue collection can be important. The Centers for Disease Control and Prevention (CDC) recommends the administration of a vaccine for Hepatitis B at birth, and in many hospitals, this vaccine is administered in the delivery room itself within minutes of a neonate's birth, often accompanied by an injection of Vitamin K. Within two months, the CDC recommends five further vaccines: RV, PTaP, Hib, PCV, and IPV. Each of these is intended to alter the neonate's responses to certain stimuli, and therefore cell collection post immunization represents a potential violation of null-exposome conditions.

Identification of Donors Meeting the Null-Exposome Conditions

While those practiced in the art of obtaining tissues associated with the birth event will already be familiar with a the normal procedures involved, including working with internal review boards (IRBs), recruiting and screening candidate donors, etc., the null-exposome conditions requirement inevitably leads to additional steps and considerations that go beyond those associated with other live tissue acquisition efforts.

For example, depending on stringency of the null-exposome conditions sought, a verification issue may arise. Many birth-related tissue collection efforts rely solely on medical information volunteered by the mother, gathered via questionnaire or interview. However, such reports are known to often contain inaccuracies, and in cases where a mother will be compensated if her child is selected as a donor, there is incentive for the mother to underreport disqualifying exposures.

Therefore, when the null-exposome conditions for a given experiment are stringent, or require abstinence from common behaviors, the practitioner must consider whether and how to undertake verification, such as by obtaining regular access to lab reports during pregnancy, or conducting post-collection screening of samples for indications of substance abuse.

The examples below are intended to further illustrate certain aspects of the methods described herein and are not intended to limit the scope of the claims.

### EXAMPLES

### Example 1: Developing a Platform for Pharmaceutical Toxicity Testing

Genetic differences among people are known to cause variations in their toxicity reactions to pharmaceuticals. However, differences in age and environment are also known to cause variations in such reactions. In this example, a scientist wants to develop a platform involving multiple human beings of both genders and various races in order to be able to test the differences in toxicity reactions to pharmaceutical compounds—differences that are due strictly to genetic differences in the test subjects, without interference from the effects of differences in the test subjects' age and environmental exposures. The scientist therefore wants to develop a set of cell lines from a cohort of human donors under null-exposome conditions.

The standard for null-exposome conditions must necessarily be quite high in order for her to succeed, as there is no comprehensive list of the exposures that might affect toxicity reactions, particularly since toxicity reactions also vary based on the compound (stimulus) in question, and the practitioner wants the platform to be open-ended—i.e., the scientist does not want to develop the platform to measure only a narrow subset of toxicity effects, or to be applicable only to a narrow subset of pharmaceutical compounds.

The scientist begins by specifying the null-exposome conditions as follows:

(1) Donor's Genetic Profile and Health: There must be no history of major genetic diseases in the families of either the donor's mother or father. The donor must be born at full-term, with birth weight between the 10th and 90th percentiles. The donor's APGAR scores must be 7 or higher at both the 1-minute and 5-minute measuring points. There must be no visible evidence of birth defects. The donor must have been exposed to no infection in utero or during the birthing process, including HIV, other sexually transmitted diseases, Hepatitis, or Strep Group B.

(2) Mother's Health History: The mother must not at any time have suffered from any disease or condition known to permanently raise the probability of birth defects in her offspring. She must not currently suffer from any of a specified list of diseases. She must not have experienced extreme malnutrition.

(3) Exposures of the Mother: Prior to and during pregnancy, the mother must not have been exposed to high levels of radiation, or to toxic substances requiring medical treatment. During pregnancy, she must not have been exposed to air or water pollution that was classified by the EPA as “high alert” status. During pregnancy, she must not have endured trauma to the abdomen requiring medical treatment.

(4) Exposures of the Fetus In Utero: During pregnancy, the mother must not have ingested high levels of alcohol, used tobacco in large quantities, taken any recreational drugs, or taken certain prescription drugs on a proscribed list.

(5) Exposures of the Neonate Post-Birth: The source tissue for the cells must be collected from the neonate immediately at birth, prior to administration of any vaccine or vitamin.

Next, the scientist utilizes the above requirements to make a choice regarding the type of perinatal cell to be used along with choices regarding selection conditions and processes.

The scientist selects as the source material Endothelial Progenitor Cells (EPCs) to be taken from umbilical cord blood. This source is preferred because the cell dates from the moment of birth (or more precisely, the moment the umbilical cord is cut) and the physical barrier of the cord protects the cells from exposure to pathogens during the birthing process and in the delivery room. The scientist considers purchasing fresh cord blood, but rules out that option as the available vendors are not prepared to support the level of patient exclusion criteria demanded by her study. Therefore, the scientist must work with one or more hospitals to obtain the necessary samples.

While the scientist is able to reach agreement with a hospital and its IRB on inclusion/exclusion criteria and collection protocols, there is a problem surrounding the issue of independent verification of maternal assertions about family histories and personal behavior during pregnancy. The scientist is eventually forced to relent on requiring independent verification of these items. Instead, the scientist opts for physical testing of the cord blood plasma to verify the absence of alcohol and drugs in the mother's system.

Finally, the remainder of the process proceeds based on established industry protocols. The EPCs are to be isolated from the cord blood according to a published protocol (Geti 2012), and then the EPCs are to be transfected into iPSCs using a commercially available kit (Stemgent, Cambridge Mass.).

### Example 2: Distinguishing Between the Effects of Two Toxicants on Persons Exposed to Both Toxicants

A toxic chemical spill has occurred in Nigeria, exposing a local tribal population to levels of a certain toxin well beyond legal limits. Numerous fatalities and nervous system injuries have resulted. The party responsible for the spill has admitted some culpability, but asserts that because the population has been exposed for many years to toxic pollution from a nearby chemical plant, some of the reported injuries are not due to the spill at all, and that any injuries resulting from the spill were made more severe by the previous long term pollution exposure. (This assertion parallels scientific findings that adverse effects from asbestos exposure are exacerbated if the victim has a history of smoking).

A practitioner is asked to conduct studies to elucidate (at least directionally) which injuries are likely due to each of the two stimuli (i.e., the recent toxic spill versus previous long term pollution exposure), and whether previous long term pollution exposure would likely have exacerbated the degree of injuries caused by the toxic spill.

The practitioner's preferred study design envisions a comparative analysis of the health and functioning of neurons under: (1) no chemical stress; (2) stress from the long term pollution only; (3) stress from the recently-spilled toxic chemical only; and (4) stress from both the long term pollution and the recently-spilled toxic chemical. To minimize any influence of genetics on the results, he chooses to utilize neurons from the same test subjects in each of the four tests, and for each of the test subjects to be members of the same tribe as the injured population. Further, in order to avoid any claims by critics that the experimental results are confounded by pre-exposure of the test subjects to historic pollution from the chemical plant or still other historical exposures, he determines that the neurons should be derived (via iPSC transfection and neuronal differentiation of those iPSCs) from null-exposome condition donors.

However, the practitioner faces constraints on his ability to collect donor cells. He is able to identify a locality whose population is of the same tribe as the injured population, and which has not been subject to significant pollution in the past. But the collection of the various types of perinatal cells is severely constrained, as: (1) late pregnancy amniocentesis is not practiced in the area, and planned Caesarian section births are rare; thus, amniotic fluid is eliminated as a source of cells; (2) skin biopsies and blood draws are judged to be too invasive for a neonate.

Therefore, the practitioner chooses to use perinatal cells taken from cord blood.

Given the genetic requirements, the practitioner specifies that: (1) five donors of each gender are required; and (2) both parents of the donor must be of the tribe specified.

In addition, the practitioner specifies a set of inclusion/exclusion health criteria for donor eligibility, including: (1) the locality and hospital are restricted to an area known to have benign air and water quality, and the mothers are to have spent the majority of their time in that locality during pregnancy; (2) the donors must be full-term neonates, as determined in this case by having a birth weight of at least 5 pounds; and (3) potential donors with a family history of certain genetic diseases are excluded.

The practitioner further specifies a set of exclusion criteria based on the behavior of the mother, where that behavior might have introduced substances with certain toxic properties that might produce similar neurological effects as the experiment is designed to measure, specifically: (1) no excessive alcohol consumption; and (2) no use of any recreational drugs or certain prescription drugs.

Of note, due to behaviors and health conditions prevalent in the local population, the practitioner does not utilize other exclusion criteria that are often employed in other attempts to create a null-exposome condition, as he judges that the impacts of such behavior would not likely affect the specific comparisons he wishes to make, but that excluding such donors would likely prevent him from obtaining enough donors in the required time period. Unused exclusion criteria here include prohibitions on tobacco use and the presence of sexually transmitted diseases that might be passed to the neonate at birth.

Once the samples of cord blood are collected, they are immediately transported to the practitioner's laboratory, where the practitioner processes the blood, cultures the required cells, and transfects them to iPSCs.. Differentiation into neurons follows well known protocols. The resulting neurons from each donor are aliquoted as appropriate, to provide the practitioner with the test subjects required for the four sets of stimulus-response studies he wishes to conduct: (1) no chemical stress (control); (2) stress from the long term pollution only; (3) stress from the recently-spilled toxic chemical only; and (4) stress from both the long term pollution and the recently-spilled toxic chemical.

### Example 3. Analyzing Drug-Drug Pharmacodynamic Interactions to Determine Whether the Degree of Reaction to the Compound of Interest (COI) Affects the Degree of Super-Additivity Between the COI and Other Compounds

A research scientist is tasked with investigating the potential for super-additive cardiotoxic pharmacodynamic effects when a particular Compound of Interest (COI) is administered to humans during the same period as certain other compounds (referred to herein as a “co-reactor”). Super-additivity is defined as synergistic effects such that the impact of the two compounds together is greater than the sum of the impacts of the two compounds acting separately. The sponsor of the research has reason to believe that any super-additivity of the particular compounds to be studied is not the same in all human beings. Specifically, the sponsor believes those individuals who exhibit a cardiotoxic reaction to the predicted therapeutic concentration of the COI (taken in isolation) that is above the 75th percentile may experience a larger degree of super-additivity when the COI is taken along with certain other compounds than those individuals having a lower than 75th percentile reaction.

The research scope is broad. For example, the sponsor is interested in the question of whether and how any increased degree of super-additivity (between the over 75th percentile group and the under 75th percentile group) changes as the dose of (either) compound is held constant while the other one is increased.

The researcher quickly eliminates from consideration the use of in vivo experiments as being either unethical (in the case of humans) or unable to examine the effects of human genetic diversity (in the case of animals). Thus, the researcher decides to conduct the work via in vitro experiments on cardiomyocytes. Primary cardiomyocytes are impractical for reasons examined earlier in this application. Thus, the researcher elects to use cardiomyocytes to be derived from iPSC cell lines from a representative sample of the population.

Further, the researcher has reason to be concerned that various environmental factors (such as smoking, certain industrial pollutants, and any history of prior treatment with chemotherapy) may have an impact on a patient's cardiotoxic reaction. However, these issues have not been previously investigated, and it is not within the scope of this particular assignment to investigate these issues. Therefore, in order to avoid the potential for confounding factors, the researcher determines to use null-exposome cells.

The researcher decides on a sample size of donors of 40. The resulting case-control sample sizes (of 10 and 30 respectively) are sufficient to support a finding of whether a distinction in super-additive behavior exists between the over and under 75th percentile cohorts. Further, from a practical standpoint, this number provides the potential for significant genetic diversity, while keeping the number of physical experiments that must be conducted low enough such that the researcher can manage the physical and data recording tasks of required by the research.

The researcher then chooses the neonatal cell type, and the criteria for donor selection that protects his required degree of null-exposome condition.

The researcher chooses to collect Endothelial Progenitor Cells from cord blood, because these cells are: (1) associated with a full term child, (2) can be obtained at the moment of birth, prior to the administration of any vaccines, etc. (3) can be collected directly from the umbilical cord via syringe, thus avoiding any exposure to air-borne contaminants, and (4) have been shown to be capable of being reprogrammed to iPSCs via non-integrating reprogramming methods that do not interfere with the DNA of the cells.

The researcher then determines the criteria for donor selection. He chooses those described above.

Cord blood collection from the donors is accomplished using processes well known to those practiced in the art. The EPCs are isolated from the cord blood and the EPCs reprogrammed into iPSCs according to the protocols referenced above.

Once the cell lines from the selected 40 donors are established, the researcher differentiates sufficient iPSC cells from each of them into cardiomyocytes using protocols described in U.S. Pat. No. 8,951,798.

The resulting cardiomyocytes are then used in cardiotoxicity assays that measure changes in heartbeat (frequency, steadiness and amplitude) using a multi-electrode array, as well as structural features (viability, mitochondrial health) using the GE Cell Health Assay. Protocols for these are well documented in the field. In each assay, the cells are challenged by a specific dose of the COI, a specific dose of one of the co-reactor or both.

The tests are organized as follows:

(1) The full set of physical experiments is specified, including the matrix of dose concentrations of each compound to be tested

(2) cardiomyocytes from all donors are prepared and aliquoted at one time into sufficient lots to support the full range of required experiments.

(3) all experiments are physical conducted in parallel, with data capture from each experiment comprising: donor identity, COI dose concentration, co-reactor dose concentration, replicate identifier, and all endpoints.

(4) The first set of data to be analyzed is from experiments wherein all donors are tested under challenge by the COI alone, at the predicted therapeutic concentration of the COI. For each endpoint, results are compared across donors to determine the (disguised) identities of those whose reactions are above versus below the 75th percentile. This work establishes the two separate cohorts (for each endpoint) that are used throughout the remainder of the data analysis.

(5) The data from the remaining physical experiments is analyzed for each endpoint being studied in the following sequence: (i) the data for experiments involving only one compound (COI, a single co-reactor) is analyzed to reveal the “stand alone” response for each endpoint for each donor at each dose. This data is then plotted on an isobologram to show the equieffective (i.e., no super-additivity) frontier. (b) Data from various COI and co-reactor mixtures are analyzed to determine the joint impact of the two compounds (at particular dose concentrations), and subsequently compared to the isobologram above to determine whether there are either sub-additive or super-additive effects. (iii) Results of steps (i) and (ii) are aggregated (into distributions) for the above 75th percentile and below 75th percentile cohorts, and those distributions compared to determine whether there is a statistically meaningful distinction.

As a result of this work, the researcher determines that there is indeed a higher likelihood of synergistic (super-additive) impact on cardiotoxicity when an individual (from the sample) takes certain of the co-reactors in conjunction with the COI, if that individual has a reaction that is above the cutoff established at the 75th percentile when the COI is taken alone at the expected therapeutic dose.

This information leads the sponsor to conduct more detailed investigations with larger sample sizes and a broader range of co-reactors. As a result of these further investigations, the sponsor requires agrees with the FDA to proscribe use of the drug developed from the COI concurrently with certain co-reactors unless a laboratory test is performed that determines that the patient has only a low level of cardiotoxic reaction to the COI itself.

### REFERENCES

Arnheim, N. and Cortopassi, G., 1992 “Deleterious mitochondrial DNA mutations accumulate in aging human tissues” Mutation Research, 275; 157-167

Arnhold, S. et al, 2011 “Amniotic Fluid Stem Cells: Growth Dynamics and Differentiation Potential after a CD-117-Based Selection Procedure” Stem Cells International, pp. 1-11

Brown, M. E. et al, 2010 “Derivation of induced Pluripotent Stem Cells from human Peripheral Blood T Lymphocytes” PLoS ONE, 5; 1-9

Cai, J. et al, 2010 “Generation of Human Induced Pluripotent Stem Cells from Umbilical Cord Matrix and Amniotic Membrane Mesenchymal Cells” The Journal of Biological Chemistry

Carraro, G. et al, 2008 “Human Amniotic Fluid Stem Cells Can Integrate and Differentiate into Epithelial Lung Lineages” Stem Cells, 26; 2902-2911

Connell, J. P. et al, 2013 “Amniotic Fluid-Derived Stem Cells for Cardiovascular Tissue Engineering Applications” Tissue Engineering: Part B, 19; 368-379

De Coppi, P. et al, 2007 “Isolation of amniotic stem cell lines with potential for therapy” Nature Biotechnology, 25; 100-106

Fakunle, E. S., 2012 “iPSCs for personalized medicine: what will it take for Africa?” Trends in Molecular Medicine, 18; 695-699

Finch, C. E., 1990 “Longevity, Senescence and the Genome” University Chicago Press, pp. 428-463

Geti, I. et al, 2012 “A Practical and Efficient Cellular Substrate for the Generation of Induced Pluripotent Stem Cells from Adults: Blood-Derived Endothelial Progenitor Cells”.

Giorgetti, A. et al, 2009 “Generation of Induced Pluripotent Stem Cells from Human Cord Blood Using OCT4 and SOX2” Cell Stem Cell, 5; 353-357

Haase, A. et al, 2009 “Generation of Induced Pluripotent Stem Cells from Human Cord Blood” Cell Stem Cell, 5; 434-441

Jiang, G. et al, 2014 “Induced Pluripotent Stem Cells from Human Placental Chorion for Perinatal Tissue Engineering Applications” Tissue Engineering, 20; 731-739

Liang, P. et al, 2013 “Drug Screening Using a Library of Human Induced Pluripotent Stem Cell-Derived Cardiomyocytes Reveals Disease-Specific Patterns of Cardiotoxicity” Circulation, 127; 1677-1691

Loh, Y., et al, 2009 “Generation of induced pluripotent stem cells from human blood” Blood, 113; 5476-5479

Mandal, P. K. and Rossi, D. J., 2013 “Reprogramming human fibroblasts to pluripotency using modified mRNA” Nature Protocol, 8; 568-582

Miller, G. W. “The Exposome, a Primer” Academic Press 2014. Chapter 1: The Exposome: Purpose, Definition, and Scope

Moretti, A. et al, 2010 “Patient-Specific Induced Pluripotent Stem-Cell Models for Long-QT Syndrome” The New England Journal of Medicine, 363; 1397-1409

Okita, K. et al, 2013 “An Efficient Nonviral Method to Generate Integration-Free Human-Induced Pluripotent Stem Cells from Cord Blood and Peripheral Blood Cells” Stem Cells, 31; 458-466

Ono, T. et al, 2002 “Mutation Theory of aging, assess in transgenic mice and knockout mice” Mechanisms of Ageing and Development, 123; 1543-1552

Park, I. et al, 2008 “Disease-Specific Induced Pluripotent Stem Cells” Cell, 134; 877-886

Quing, L. I. et al, 2013 “Generation of Induced Pluripotent Stem Cells from Human Amniotic Fluid Cells by Reprogramming with Two Factors in Feeder-free conditions” Journal of Reproduction and Development, 59; 72-77

Steigman, S. A. et al, 2008 “Preclinical regulatory validation of a 3-stage amniotic mesenchymal stem cell manufacturing protocol” Journal of Pediatric Surgery, 42; 1164-1169

Trifunovic, A. and Larsson, N. G., 2008 “Mitochondrial dysfunction as a cause of ageing” Journal of Internal Medicine, 263; 167-178

Vijg, J., 2000 “Somatic mutations and aging; a re-evaluation.” Mutation Res., 447; 117-135

Wang, J. et al, 2013 “Generation of Induced Pluripotent Stem Cells with High Efficiency from Human Umbilical Cord Blood Mononuclear Cells” Genomics Proteomics Bioinformatics, 11; 304-311

Warren, L. et al, 2010 “Highly Efficient Reprogramming to Pluripotency and Directed Differentiation of Human Cells with Synthetic Modified mRNA” Cell Stem Cell, 7; 618-630

Wild, C., 2005 “Complementing the genome with an “exposome”: the outstanding challenge of environmental exposure measurement in molecular epidemiology.” American Association for Cancer Research, pp. 1847-1849

You, Q. et al, 2008 “Isolation of human mesenchymal stem cells from third-trimester amniotic fluid” International Journal of Gynecology and Obstetrics, 103; 149-152

Zhou, T. et al, 2012 “Generation of human induced pluripotent stem cells from urine samples” Nature Protocol, 7; 2081-2088

The methods of the appended claims are not limited in scope by the specific methods described herein, which are intended as illustrations of a few aspects of the claims and any methods that are functionally equivalent are within the scope of this disclosure. Various modifications of the methods in addition to those shown and described herein are intended to fall within the scope of the appended claims. Further while only certain representative methods and aspects of these methods are specifically described, other methods and combinations of various features of the methods are intended to fall within the scope of the appended claims, even if not specifically recited. Thus, a combination of steps, elements, components, or constituents may be explicitly mentioned herein; however, all other combinations of steps, elements, components and constituents are included, even though not explicitly stated. All publications, patents, and patent applications cited herein are hereby incorporated by reference in their entireties for all purposes.

1. A method for determining a response of a test sample to a stimulus, comprising
a) applying the stimulus to the test sample, wherein the test sample comprises one or more induced pluripotent stem cells, or one or more cells differentiated therefrom, and the induced pluripotent stem cells have been derived from perinatal cells isolated from one or more donors, wherein the perinatal cells have been isolated from one or more donors under null-exposome conditions and
b) detecting the response by the test sample to the stimulus, wherein detection of a response, when compared to an unstimulated control, indicates that the stimulus has an effect on the test sample.

2. The method of claim 1, wherein the donors are human.

3. The method of claim 1, wherein the stimulus is a chemical.

4. The method of claim 3, wherein the chemical is a pharmaceutical compound.

5. The method of claim 1, wherein the stimulus is a biological agent.

6. The method of claim 1, wherein the differentiated cells form one or more tissues.

7. The method of claim 1, wherein the number of donors is ten or more.

8. The method of claim 1, wherein the response is quantified.

9. A method of determining the effect of genetic differences on a response of a plurality of test samples to a stimulus, comprising
a) applying the stimulus to the test samples, wherein each test sample comprises one or more induced pluripotent stem cells, or one or more cells differentiated therefrom, and the induced pluripotent stem cells have been derived from perinatal cells isolated from one or more donors, wherein the perinatal cells have been isolated from one or more donors under null-exposome conditions, and
b) detecting the response by the test samples to the stimulus, wherein detection of a response, when compared to other test samples, indicates that the stimulus has an effect on the test samples due to the genetic differences of the samples.

10. The method of claim 9, wherein the perinatal cells have been isolated from one or more donors under null-exposome conditions.

11. The method of claim 9, wherein the donors are human.

12. The method of claim 9, wherein the stimulus is a chemical.

13. The method of claim 12, wherein the chemical is a pharmaceutical compound.

14. The method of claim 9, wherein the stimulus is a biological agent.

15. The method of claim 9, wherein the differentiated cells form one or more tissues.

16. The method of claim 9, wherein the number of donors is ten or more.

17. The method of claim 9, wherein the response is quantified.
